(19) United States
(12) Patent Application Publication
(10) Pub. No.: US 2025/0101414 A1
(43) Pub. Date: Mar. 27, 2025

(54) PARTICLE-TEMPLATED EMULSIFICATION IN WELL PLATES

(71) Applicants: CZ BIOHUB SF, LLC, San Francisco, CA (US); THE REGENTS OF THE UNIVERSITY OF CALIFORNIA, Oakland, CA (US)

(72) Inventors: Adam Abate, Okland, CA (US); Daniel Weisgerber, Oakland, CA (US)

(21) Appl. No.: 18/727,209

(22) PCT Filed: Jan. 9, 2023
(86) PCT No.: PCT/US2023/010423

§ 371 Date: Jul. 8, 2024

Related U.S. Application Data
(60) Provisional application No. 63/298,043, filed on Jan. 10, 2022.

(51) Int. Cl. C12N 15/10

(52) U.S. Cl. CPC C12N 15/1075; C12N 15/1065

(57) ABSTRACT

The present disclosure provides materials and methods for producing droplet libraries with thousands of distinct reagents. The monodispersed libraries prepared according to the methods provided herein use multiwall plates and are compatible with microfluidic processing.

### CROSS-REFERENCE TO RELATED APPLICATIONS

The present application claims priority to U.S. Provisional Patent Application No. 63/298,043, filed on Jan. 10, 2022, their entirety of which are incorporated by reference herein.

### STATEMENT REGARDING FEDERALLY SPONSORED RESEARCH

This invention was made with government support under grants AI129206 and R01 EB019453 awarded by The National Institutes of Health, grant 75D301-21-R-71738 awarded by Centers for Disease Control and Prevention, and grant N66001-18-C-4507 awarded by The Defense Advanced Research Projects Agency. The government has certain rights in the invention.

### FIELD

The present disclosure relates generally to methods for generating diverse droplet libraries for applications that, previously, had been impractical with microfluidics.

### BACKGROUND

Droplet microfluidics uses microscale channels to encapsulate and process samples in emulsions (S.-Y. Teh, et al., Lab Chip, 2008, 8, 198; S. Mashaghi, et al., Trends in Analytical Chemistry, 2016, 82, 118-125; and L. Shang, Y. et al., Chem. Rev., 2017, 117, 7964-8040). Some applications require droplet libraries, emulsions consisting of thousands of droplet types containing distinct chemical or biological reagents (E. Brouzes, et al., Proceedings of the National Academy of Sciences, 2009, 106, 14195-14200; and T. S. Kaminski, et al., Lab Chip, 2012, 12, 3995) For example, drug or antibiotic screens require that a drug library be encapsulated in picoliter droplets to process it with microfluidic devices (A. Kulesa, et al., Proc Natl Acad Sci USA, 2018, 115, 6685-6690). Alternatively, combinatorial chemical synthesis requires mixing thousands of distinct precursors to generate millions or billions of reaction products (O. N. Burchak, et al., J. Am. Chem. Soc., 2011, 133, 10058-10061). While of high value, these applications are impractical with droplet microfluidics due to the challenges in creating droplet libraries (K. R. Bajgiran, et al., Micromachines, 2021, 12, 1211).

The most common approach for generating a droplet library is to emulsify each sample in a separate microfluidic device, then pool the results (A. Kulesa, et al., Proc Natl Acad Sci USA, 2018, 115, 6685-6690; O. N. Burchak, et al., J. Am. Chem. Soc., 2011, 133, 10058-10061). Laborious for just tens of reagents, this approach is impractical for the thousands needed for combinatorial applications. While it can be automated using sequential tube emulsification, this hardly increases scalability, and runs the risk of cross contamination since all reagents flow through the same injection tube (A. B. Theberge, et al., Lab Chip, 2012, 12, 1320). Alternatively, parallel approaches can emulsify tens of samples in separate devices and interface with well plates; however, the devices have complex architectures that are hard to fabricate and prone to failure due to defects or dust, often yielding inferior emulsions ((K. R. Bajgiran, et al., Micromachines, 2021, 12, 1211; and X.-H. Ji, et al., Chem., 2011, 21, 13380). Recently, commercial liquid dispensers capable of automatically processing well plates have generated droplet libraries of hundreds of reagents (J. Q. Zhang, et al., Sci Rep, 2021, 11). The instrument can controllably aspirate and dispense from a micron-scale glass capillary to emulsify each sample. Automation allows the machine to run hands-free for hours to emulsify hundreds of samples, and the capillary can be rinsed between reagents to minimize cross contamination. Nevertheless, scaling to thousands is impractical due to the sequential processing, and cross contamination is still possible because one capillary is used to process all samples. To generate libraries containing thousands of different droplets, a new approach is needed.

### SUMMARY OF THE INVENTION

One embodiment of the present disclosure provides a method of generating a droplet library, comprising: (a) preparing a reagent library comprising combining in single wells of a multi-well plate (i) a collection of particles; (ii) an immiscible carrier; and (iii) a solution comprising a plurality of reagents; (b) agitating the combined solution of (a) under conditions that allow the encapsulation of the solution comprising a plurality of reagents into droplets, thereby forming a monodisperse emulsion; and (c) optionally pooling the droplets formed in the individual wells of (b) into a vessel; thereby generating a droplet library. In some embodiments, the multi-well plate comprises 4, 6, 8, 12, 24, 48, 96, 384, or 1536 wells.

In other embodiments, an aforementioned method is provided wherein the total aqueous volume in an individual well is between approximately 3-100 μl. In other embodiments, an aforementioned method is provided wherein the droplet diameter is approximately 20-200 um. In another embodiment, the multi-well plate comprises 96 wells, wherein the total aqueous volume is approximately 200 μl, and wherein the droplet diameter is approximately 60-70 um. In still another embodiment, the multi-well plate comprises 384 wells, wherein the total aqueous volume is approximately 10 μl, and wherein the droplet diameter is approximately 60-70 um. In yet another embodiment, the multi-well plate comprises 1536 wells, wherein the total aqueous volume is approximately 3 μl, and wherein the droplet diameter is approximately 60-70 um.

In other embodiments of the present disclosure, an aforementioned method is provided wherein the particles are selected from the group consisting of hydrogel beads, plastic beads, glass beads, ceramic beads, and magnetic beads. In another embodiment, the particles are hydrogel beads comprising acrylamide. In other embodiments, an aforementioned method is provided wherein the immiscible carrier is an oil. In one embodiment, the oil comprises a fluorosurfactant and N,N,N,N-tetramethylethylenediame in hydrofluoroether. In other embodiments, an aforementioned method is provided wherein prior to agitating, the multi-well plate is sealed. In another embodiment, the agitating comprises mixing the reagents by pipetting, shaking by hand, stirring, beating, bubbling, vortexing and sonicating.

In still other embodiments, an aforementioned method is provided wherein each well of the multi-well plate comprises a solution comprising a plurality of reagents and wherein at least one reagent is unique to each individual well. In other embodiments, an aforementioned method is provided wherein the plurality of reagents comprises a sample comprising biomolecules. In another embodiment, the sample is obtained from a human subject. In some embodiments, the sample is a saliva, blood, urine, or tissue sample. In still another embodiment, the sample comprises a plurality of cells selected from the group consisting of a virus or virus particle, a bacterial cell, a yeast cell, a parasitic cell, or a human cell. In yet another embodiment, the sample has not undergone purification steps prior to combining in a well of step (a).

The present disclosure, in another embodiment, provides an aforementioned method wherein the plurality of reagents comprises at least one nucleic acid. In another embodiment, the plurality of reagents comprises reagents suitable for amplifying the nucleic acid. In still another embodiment, the plurality of reagents comprises reagents suitable for a polymerase chain reaction (PCR) or reagents suitable for a loop-mediated isothermal amplification (LAMP) reaction or reagents for a nucleic acid sequence-based amplification (NASBA) reaction, and optionally comprising a lysing reagent. In yet another embodiment, the lysing reagent, when present, is SDS. In other embodiments, the droplet is incubated under conditions that allow amplification, and the nucleic acid is amplified by a method selected from the group consisting of PCR, RT-PCR, qPCR, digital droplet PCR (ddPCR), LAMP and NASBA.

In other embodiments, an aforementioned method is provided wherein each droplet formed in step (b) comprises a single cell. In other embodiments, an aforementioned method is provided wherein each droplet comprises a barcode. In still other embodiments, an aforementioned method is provided further comprising the step of removing satellite droplets. In other embodiments, an aforementioned method is provided further comprising the step of sorting the droplets.

### BRIEF DESCRIPTION OF THE DRAWINGS

FIG. 1 shows the scalable production of droplet libraries is achievable using well plate particle-templated emulsification. (Left) The reagent library is assembled in a well plate. Within each well, templating particles, reagents, and oil are added respectively. Importantly, each well contains a unique sample for emulsification. (Middle) Agitation by vortexing the plate agitates each well. This results the emulsification of reagents within each well. Sealing the plate prevents any cross contamination between wells during agitation. (Right) Following emulsification, the droplets are pooled from the well plate to form the droplet library.

FIGS. 2A-2D show uniform droplet emulsification using plate-based particle-templated emulsification. (FIG. 2A) Droplet size was evaluated in a 384 well plate. There is no significant difference throughout a 384 well plate. Using plate particle-templated emulsification, we can also scale droplet libraries for increased volume or diversity. (FIG. 2B) For 96 well plates we generate 4× more droplets than in a 384 well plate. (FIG. 2C) For 1536, 4× the number of emulsions for the library is generated. (FIG. 2D) No significant difference in droplet size between well plates was observed, suggesting droplet size depends on templating particle.

FIGS. 3A-E show removal of satellites characteristic of particle-templated emulsification by sequential oil washes. (FIG. 3A) Following creaming, droplets floating to the oil-air interface, removal of the bottom oil layer will remove satellites. Repeating this oil wash removes an increasing fraction of satellites. (FIG. 3B) After 30 seconds a distinct layer of PTE droplets is identifiable. Partial to complete removal of the oil layer below this removes satellites. This successful removal is observable by the reduction in oil opacity. (FIG. 3C) Microscopic imaging confirms the reduction in satellites. This is especially apparent at higher magnifications after three washes. (FIG. 3E) A quantification at high magnification identifies that 99% of satellites are removed after three washes.

FIG. 4A-4B show fluorescent droplet library prepared with plate particle-templated emulsification. (FIG. 4A) A 384 well plate to generate a droplet library containing 16 different emulsions. These emulsions consist of two dyes (Cy5 and Cascade Blue) mixed at four different concentrations. (FIG. 4B) The fluorescent profiles of individual wells were measured to verify 16 distinct emulsions. All wells were pooled and measure individual droplet fluorescence in the droplet library. This confirms the successful generation a droplet library using plate PTE.

FIGS. 5A-5D show washing and reinjection of plate emulsified droplets. (FIG. 5A) Microfluidic reinjection and sorting of particle-templated emulsification. (FIG. 5B) Reinjection of droplets into microfluidic device. (FIG. 5C) Separation of droplets via bias oil within microfluidics. Sorting (FIG. 5D) positive and negative droplets library based on fluorescence detection.

FIGS. 6A-6C show well plate templated emulsification supports droplet digital PCR reactions. (FIG. 6A) A series of dilutions (1×, 0.02×, 0.002×, 0.0002×, 0.00002×, 0.000002×, 0.0000002×, and 0×) was prepared and added to a 384 well plate for templated emulsification. (FIG. 6B) A measure of the fluorescent signal from the droplet digital PCR reaction quantifies the fraction of positive droplets. (FIG. 6C) A calculation of copes per μL determined and the associated Poisson estimator calculated as λ=1/nΣᵢ₌₁ⁿkᵢ with n total and kᵢ values.

FIGS. 7A-7C shows the generation of double emulsions using particle templated emulsification. (FIG. 7A) Particles used in PTE to generate emulsions; (FIG. 7B) Single emulsions generated using PTE; and (FIG. 7C) Conversion to double emulsions using single PTE emulsions.

FIGS. 8A-8E show PTE double emulsions size and stability is affected by droplet size. (FIG. 8A) Initial generation of PTE double emulsions results in a relatively thin oil layer separating the inner aqueous droplet contents from the outer aqueous phase. (FIG. 8B) Addition of salt to the outer buffer results in a hypertonic solution causing the double emulsion to shrink. (FIG. 8C) Further addition of salt to the outer solution causes the droplet to continue to shrink. The smaller double emulsions are significantly more stable than the original emulsions. (FIG. 8D and FIG. 8E) For PTE double emulsions, shrinking the double emulsion results in a significantly brighter signal.

FIGS. 9A-9C show PTE double emulsions can be sorted via FACS and avoids the use of custom made microfluidic sorters. (FIG. 9A) FACS side scatter vs FITC identifies two unique populations of droplets. (FIG. 9B) distinct populations of FITC positive (ddPCR positive) and FITC negative double emulsions can be identified via FACS. (FIG. 9C) Additionally, fractions of double emulsions containing single, double, and triple cores can be identified using FACS. Gating and sorting based on these populations is possible using the FACS.

### DETAILED DESCRIPTION

The present disclosure addresses the aforementioned need in the art and provides a bulk approach for producing droplet libraries with thousands of distinct reagents. The monodispersed libraries are compatible with microfluidic processing. The methods described herein affords a supremely scalable means for generating diverse droplet libraries for applications that, previously, had been impractical with microfluidics.

The present disclosure provides, in some embodiments, a simple, scalable, and cross-contamination free approach for generating high diversity droplet libraries. The approach uses Particle-templated Emulsification (PTE) to encapsulate thousands of samples simultaneously by bulk agitation of a well plate (M. N. Hatori, et al., Anal. Chem., 2018, 90, 9813-9820; and US Pub. 2020/0261879, incorporated by reference herein) The resultant emulsions are monodispersed and pooled to generate a droplet library. Because the samples are emulsified in separate wells, cross contamination is avoided entirely. In some embodiments, the methods described herein use standard well plates of common available sizes (96, 384, and 1536), and a plate vortexer to generate the emulsions. Hence, a single round can generate over a thousand emulsions in ˜30 s. Accounting for pipetting of oil, surfactant, particles, and library pooling, the methods provided herein allow generation of libraries comprising tens of thousands of distinct regents in a few hours. Plate PTE thus affords a simple means by which to generate massive emulsion libraries for combinatorial applications that have previously been impractical in droplet microfluidics, greatly expanding the applicability of this important technology.

The present disclosure provides an improved particle-templated emulsification (PTE) method for generating monodisperse emulsions. The droplets present in such emulsions are referred to interchangeably herein as PTE droplets. The disclosed methods facilitate the encapsulation and subsequent analysis of target particles of interest. The disclosed methods involve the use of monodisperse particles to template the formation of monodisperse droplets. In some embodiments, the disclosed methods facilitate the encapsulation of target particles, e.g., nucleic acids, which can then be detected, quantitated and/or sorted, e.g., based on their sequence as detected with nucleic acid amplification techniques, e.g., PCR and/or MDA.

Particle-templated emulsification (PTE) allows for separation and encapsulation of samples during vortexing. See, e.g., WO/2019/139650 and US Pub. 2020/0261879, incorporated by reference in its entirety herein. However, while the aforementioned method generates a monodisperse emulsion that encapsulates target particles of interest without requiring the use of a microfluidic device, this method usually generates satellite droplets that may cause template loss and quantification bias.

In one embodiment, the a method of preparing a droplet, a population of droplets, or a library of droplets, is provided herein. A “droplet library” as used herein refers to a collection of droplets containing distinct reagents prepared from a reagent library by the emulsification of each distinct reagent separately.

As described herein, a “reagent library” refers to any number of distinct reagents, typically 10-10,000 in number, that are to be emulsified separately to avoid cross contamination. Examples include drugs, chemicals, or cells.

In some droplet-based multivolume techniques, the method requires fabrication of microfluidic chips with multi-volume chambers or micropillar with multi-sized surface areas (See, e.g., Liu, W-W., et al., Anal. Chem. 2017, 89, 822-829; Shen, F., et al., J. Am. Chem. Soc., 2011, 133, 17705-17712; and Kreutz, J., et al., Anal. Chem., 2011, 83, 8159-8168).

As used herein, the term “sample” or “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, nucleic acids, proteins serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual.

As used herein, the term “monodisperse,” as applied to particles or droplets, refers to a variation in diameter or largest dimension of the particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01. In some embodiments, monodisperse droplets have a diameter of about 1.0 pm to 1000 pm, inclusive, such as about 1.0 pm to about 750 pm, about 1.0 pm to about 500 pm, about 1.0 pm to about 250 pm, about 1.0 pm to about 200 pm, about 1.0 pm to about 150 pm, about 1.0 mih to about 100 mih, about 1.0 mih to about 10 mih, or about 1.0 mih to about 5 mhi, inclusive. In some embodiments, the internal volume of the monodisperse droplets may be about 0.01 pL or less, about 0.1 pL or less, 1 pL or less, about 5 pL or less, 10 pL or less, 100 pL or less, or 1000 pL or less. In some embodiments, the internal volume of the monodisperse droplets may be about 1 fL or less, about 10 fL or less, or 100 fL or less. In some embodiments, the internal volume of the monodisperse droplets may encompass a liquid volume which ranges between picoliters and femotliters (e.g., about 0.001 pL to about 1000 pL). In some embodiments, the internal volume of the monodisperse droplets extends at the nanoliter level or below the nanoliter level (e.g., strictly picoliter, strictly femtoliter, or combination thereof). Thus, the term “polydisperse” refers to an emulsion comprising non-uniform particles or droplets with unequal sizes.

As described herein, the present detection methods include droplets, e.g., droplets of oil, of varying sizes. In some embodiments, droplet size is approximately between 1 um and 1000 um, where the corresponding volume ranges would be approximately 0.004 pL (1 um dia drop) to 4000 nL (1000 um dia drop). In one embodiment, the droplets are between approximately 10 μm and 300 μm or 20 μm and 200 μm in size. In another embodiment, the droplets are between approximately 50 μm and 120 μm in size. In some embodiments, the droplet diameter is between approximately 20-200 um. In some embodiments, the multi-well plate comprises 4, 6, 8, 12, 24, 48, 96, 384, or 1536 wells. In various embodiments, the total aqueous volume in an individual well is between approximately 3-100 μl. In general, the repent methods provide that the aqueous volume is ¼ working volume-for example, 1536 uses 3 uL, 384 uses 10 uL, 96 uses 100 uL; oil volume is ½ working volume and air volume is ¼ working volume.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA. In particular, the polynucleotides and nucleic acids of the present disclosure refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

In various embodiments, a fluorescent profile that is produced by fluorophores is detected as part of the methods disclosure herein. As used herein, “fluorescent profile” means the fluorescent signal comprised of one or more wavelengths generated through the excitation of specific/defined concentrations and combinations of fluorescent probes used to identify a specific target. Various means of detecting fluorsecene or fluorescent profiles are known in the art and are provided herein, including, for example fluorescent microscopy, FACS, microfluidic readers, and droplet readers. Generally, any means of fluorescent microscopy can be applied. FACS (fluorescence activated cell sorting), for example, is possible with, for example, conversion to double emulsions. Reinjection of droplets into a microfluidic device using optics to fluorescently detect/analyze the droplets is also contemplated. Droplet readers (instruments like BioRad QX200) are microfluidic devices capable of detecting droplet fluorescence for analysis and are also contemplated herein.

Some methods of the present disclosure include particles that provide monodispersed emulsions. As described herein, particles include, but are not limited to, hydrogel beads, plastic beads, glass beads, ceramic beads, and magnetic beads. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), polyacrylamide/poly (acrylic acid) (PAA), hydroxyethyl methacrylate (HEMA), poly N-isopropyl acrylamide (NIP AM), and polyanhydrides, polypropylene fumarate) (PPF).

According to some embodiments of the present disclosure, a lysing reagent is used in the methods. Lysing agents may include, for example chemical lysis, such as SDS, detergents, alkaline, and acid; biological lysis, such as lysis enzymes, viruses, and phages; and physical lysis such as beads beating, grinding, frozen-thaw, and sonication.

In certain aspects, a surfactant may be included in the vessels (i.e., wells of a multiwall plate) and methods described herein. Accordingly, a droplet may involve a surfactant stabilized emulsion, e.g., a surfactant stabilized single emulsion or a surfactant stabilized double emulsion. Any convenient surfactant that allows for the desired reactions to be performed in the droplets may be used, including, but not limited to, octylphenol ethoxylate (Triton X-100), polyethylene glycol (PEG), C26H50010 (Tween 20) and/or octylphenoxypolyethoxyethanol (IGEPAL). In other aspects, a droplet is not stabilized by surfactants. The surfactant used depends on a number of factors such as the oil and aqueous phases (or other suitable immiscible phases, e.g., any suitable hydrophobic and hydrophilic phases) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to a hydrocarbon oil, for example, the surfactant may instead be chosen such that it had a hydrophobic hydrocarbon block, like the surfactant ABIL EM90.

Other surfactants can also be envisioned, including ionic surfactants. Other additives can also be included in the oil to stabilize the droplets, including polymers that increase droplet stability at temperatures above 35° C. Without intending to be bound by any particular theory, it is proposed that the preparation of a thermostable emulsions relies on the use of a surfactant that is able to form membranes or double emulsion interfaces that can optionally withstand high temperatures, such as those associated with standard PCR reactions. One way to accomplish this may be to use a surfactant with a relatively high molecular weight so that when assembled at the interface of a droplet or in a membrane configuration, the energy required to remove the surfactant from the interface (or break the membrane) is higher than can be provided by kT. Exemplary surfactants which may be utilized to provide thermostable emulsions are the “biocompatible” surfactants that include PEG-PFPE (polyethyleneglycol-perflouropolyether) block copolymers, e.g., PEG-Krytox® (see, e.g., Holtze et ak, “Biocompatible surfactants for water-in-fluorocarbon emulsions,” Lab Chip, 2008, 8, 1632-1639, the disclosure of which is incorporated by reference herein), and surfactants that include ionic Krytox® in the oil phase and Jeffamine® (polyetheramine) in the aqueous phase (see, e.g., DeJoumette et ak, “Creating Biocompatible Oil-Water Interfaces without Synthesis: Direct Interactions between Primary Amines and Carboxylated Perfluorocarbon Surfactants”, Anal. Chem. 2013, 85(21): 10556-10564, the disclosure of which is incorporated by reference herein). Additional and/or alternative surfactants may be used provided they form stable interfaces. Many suitable surfactants will thus be block copolymer surfactants (like PEG-Krytox®) that have a high molecular weight. These examples include fluorinated molecules and solvents, but it is likely that non-fluorinated molecules can be utilized as well.

The present disclosure provides methods of detecting a target in a sample, where the target may be, for example, a nucleic acid (RNA, DNA), biomolecules such nucleic acids, genes, proteins or polypeptides or epitopes, as well as biological particles such as cells (bacterial, human, parasite) and viruses.

Exemplary pathogenic bacteria or bacterial cells include, for example, members of the genus Actinomyces, Bacillus, Bacteroides, Bordetella, Bartonella, Borrelia (e.g., B. burgdorferi OspA), Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus polypeptides, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus polypeptides (i.e., S. pneumoniae polypeptides), Proteus, Pseudomonas, Rickettsia, Rochalimaea, Salmonella, Shigella, Staphylococcus, group A Streptococcus (e.g., S. pyogenes), group B Streptococcus (S. agalactiae), Treponema, and Yersinia.

Exemplary pathogenic viruses or virus particles or viral genomes include, for example, adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In cetain embodiments, the virus is SARS-COV-2, HIV, HSV, or HPV.

Exemplary parasites include protozoan parasites, for example, members of the Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca Spirometra, Stephanofilaria, Strongyloides, Strongylus, Thelazia, Toxascaris, Toxocara, Trichinella, Trichostrongylus, Trichuris, Uncinaria, and Wuchereria. Pneumocystis, Sarcocystis, Schistosoma, Theileria, Toxoplasma, and Trypanosoma are also contemplated.

Various embodiments of the present disclosure use a polymerase chain reaction (PCR)-based assay (e.g., as an optional step with the methods provided herein). Examples of PCR-based assays of interest include, but are not limited to, quantitative PCR (qPCR), quantitative fluorescent PCR (QF-PCR), multiplex fluorescent PCR (MF-PCR), digital droplet PCR (ddPCR) single cell PCR, PCR-RFLP/real time-PCR-RFLP, hot start PCR, nested PCR, in situ polony PCR, in situ rolling circle amplification (RCA), bridge PCR, picotiter PCR, emulsion PCR and reverse transcriptase PCR (RT-PCR). Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABS A). A PCR-based assay may be used to detect the presence of certain nucleic acids or gene(s). In such assays, one or more primers specific to each gene of interest are reacted with the genome of each cell. To determine whether a particular gene is present, the PCR products may be detected through an assay probing the liquid of a droplet as described herein, such as by staining the solution with an intercalating dye, like SybrGreen or ethidium bromide, hybridizing the PCR products to a solid substrate, such as a bead (e.g., magnetic or fluorescent beads, such as Luminex beads), or detecting them through an intermolecular reaction, such as FRET. These dyes, beads, and the like are each examples of a “detection signal,” a term that is used broadly and genetically herein to refer to any component that is used to detect the presence or absence of nucleic acid amplification products, e.g., PCR products.

Loop-mediated isothermal amplification (LAMP) and digital droplet LAMP (ddLAMP) is a single-tube technique for the amplification of DNA and a low-cost alternative to detect certain diseases and is also contemplated herein. Reverse Transcription Loop-mediated Isothermal Amplification (RT-LAMP) combines LAMP with a reverse transcription step to allow the detection of RNA. LAMP is an isothermal nucleic acid amplification technique. In contrast to the polymerase chain reaction (PCR) technology, in which the reaction is carried out with a series of alternating temperature steps or cycles, isothermal amplification is carried out at a constant temperature, and does not require a thermal cycler. In LAMP, the target sequence is amplified at a constant temperature of 60-65° C. using either two or three sets of primers and a polymerase with high strand displacement activity in addition to a replication activity. Typically, 4 different primers are used to amplify 6 distinct regions on the target gene, which increases specificity. An additional pair of “loop primers” can further accelerate the reaction. The amount of DNA produced in LAMP is considerably higher than PCR-based amplification. LAMP and RT-LAMP (<30 min) is much faster than PCR-based assays (>1 h).

In various embodiments, the methods provided herein use an immiscible carrier. Immiscible carriers, which form a non-aqueous phase in the emulsions described herein, include but are not limited to a fluorocarbon oil, a hydrocarbon oil, or a combination thereof; and the third fluid is an aqueous phase fluid. The non-aqueous phase may serve as a carrier fluid forming a continuous phase that is immiscible with water, or the non-aqueous phase may be a dispersed phase. The non-aqueous phase may be referred to as an immiscible carrier or oil phase including at least one oil, but may include any liquid (or liquefiable) compound or mixture of liquid compounds that is immiscible with water. The oil may be synthetic or naturally occurring. The oil may or may not include carbon and/or silicon, and may or may not include hydrogen and/or fluorine. The oil may be lipophilic or lipophobic. In other words, the oil may be generally miscible or immiscible with organic solvents. Exemplary oils may include at least one silicone oil, mineral oil, fluorocarbon oil, vegetable oil, or a combination thereof, among others. In exemplary embodiments, the oil is a fluorinated oil, such as a fluorocarbon oil, which may be a perfluorinated organic solvent. Examples of a suitable fluorocarbon oils include, but are not limited to, C9H5OF15 (HFE-7500), C21F48N2 (FC-40), and perfluoromethyldecalin (PFMD).

A variety of different detection components may be used in practicing the subject methods, including using fluorescent dyes known in the art. Fluorescent dyes may typically be divided into families, such as fluorescein and its derivatives; rhodamine and its derivatives; cyanine and its derivatives; coumarin and its derivatives; Cascade Blue and its derivatives; Lucifer Yellow and its derivatives; BOD IP Y and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, VA. In some embodiments, the detection or quantification methods described herein use a combination of different detection components. By way of example, different fluorophores can be conjugated to primers that allow the detection of intact/whole viral genomes versus fragmented viral genomes as described herein.

In one embodiment, the methods comprise removing oil droplets, e.g., of a particular size. Removal of satellite droplets is achieved via consecutive oil washes. Oil washes consist of mixing the sample (droplets+satellites) and waiting a fixed time (30 sec-1 min) for droplets to settle (cream). Because of the dependency on volume, larger droplets will settle (cream) before the smaller satellite droplets. Removal of the bottom oil layer before the satellites settle (cream) will remove that proportion of the satellites. The oil volume is then replaced with fresh oil and the process repeated. Consecutive washes remove additional satellites, with 3 consecutive washes removing up to 99% of satellites. Satellite droplets are small droplets that appear, in the case of PTE, as a byproduct of agitation (vortexing).

In other embodiments, drug screening is provided herein. Drug screening is accomplished by preparing droplet libraries within well plates then merging these droplets with a similarly prepared droplet library of cells. For the drug library, hundreds to thousands of different drugs can be individually encapsulated using plate PTE. Similarly, a single cell target or hundreds to thousands of cell targets can be individually encapsulated using PTE. Using a microfluidic device, droplets from the cell target library and drug library can be combinatorially combined allowing for thousands to millions of different combinations, testing each cell with one or more drugs.

In still another embodiment, conversion of PTE drops described herein are converted to double emulsions is provided herein. For example, a conversion from water-in-oil to water-in-oil-in-water is provided, allowing for the manipulation of the droplet library in aqueous mediums, such as sorting with FACS.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLE 1

### Particle-Templated Emulsification in Well Plates

Particle-templated emulsification generates droplets by breaking a continuous aqueous phase into consecutively smaller droplets by agitation (M. N. Hatori, Set al., Anal. Chem., 2018, 90, 9813-9820). The hydrogel templating particles in the aqueous phase inhibit breakup of droplets smaller than the particle diameter such that, if the particles are monodispersed, the final droplets are also monodispersed, with a size slightly larger than the particles. The sample is encapsulated within and around the permeable hydrogel particle residing in each final droplet. Hence, final droplet size can be controlled with particle size (M. N. Hatori, Set al., Anal. Chem., 2018, 90, 9813-9820). Alternatively, because emulsification occurs for all droplets in parallel and takes a total of 30 s for all samples, the amount of produced emulsion scales with sample volume rather than emulsification time as in a conventional microfluidic device, making emulsions comprising thousands to trillions of uniform droplets producible in seconds.

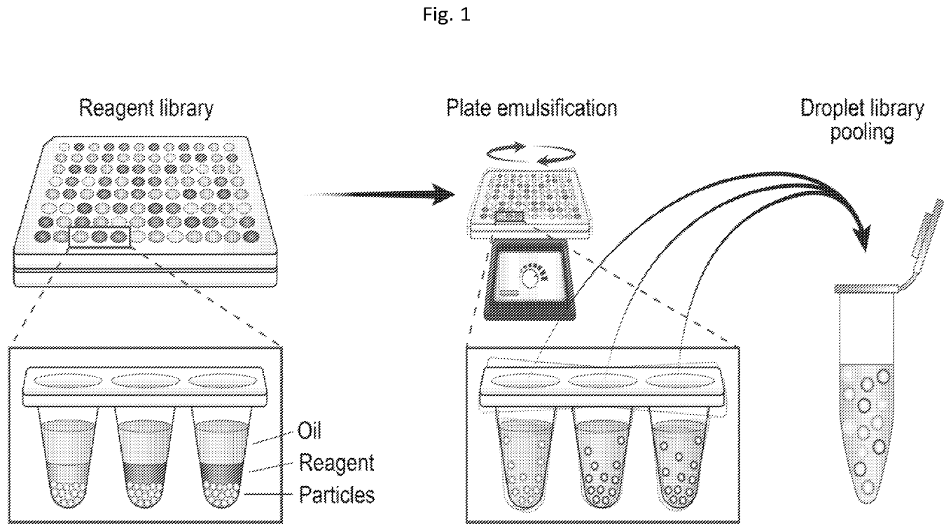

The innovation of this paper is to recognize that because each sample is emulsified in its own tube, PTE can scale up the production of emulsions by parallelization in well plates. To form a library, the emulsions generated in the separate wells are combined by pipetting. To implement the approach, each well is loaded with a unique reagent, templating particles, and surfactant-laden oil (FIG. 1, left). After preparation of all wells, the plate is agitated on a plate vortexer, injecting the energy that induces PTE in each well (FIG. 1, center). To enable sufficient agitation, an air gap at the top of each well is necessary to supply sufficient space for the oil-sample mixture to flow. We have found that a reagent: oil: air volume ratio of 1:2:1 supplies a sufficient air gap for emulsification. In a 384 well plate, with a working volume of 40 μL per well, this allows 8 μL of hard-packed beads and 2 μL of sample per well. Following 30 seconds of agitation, pooling the wells results in the droplet library (FIG. 1, right).

### Characterization of Plate-Based Particle-Templated Emulsification

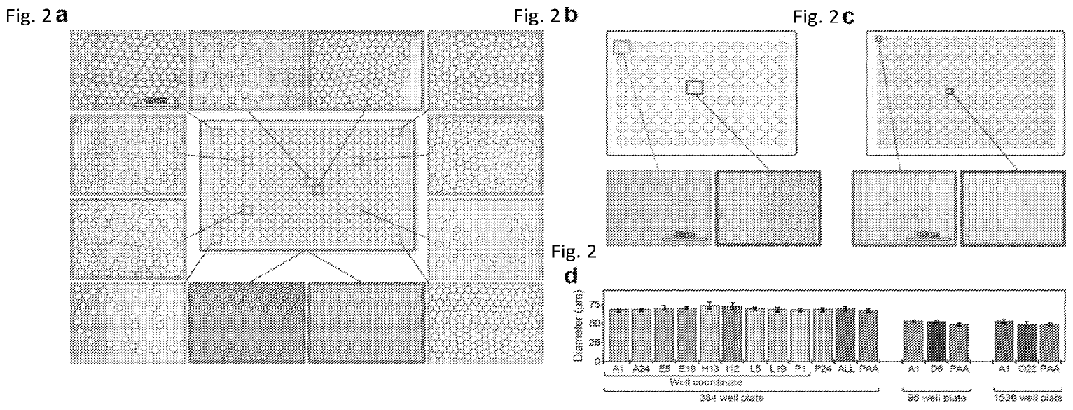

To process droplets with microfluidic devices, they must be monodispersed so that the channels can be properly sized for actions like spacing, merging, and sorting (N. M. Kovalchuk, et al., Chemical Engineering Research and Design, 2018, 132, 881-889; C. Sommer, et al., Lab Chip, 2014, 14, 2319-2326; and C. N. Baroud, et al., Lab Chip, 2010, 10, 2032). To confirm that plate PTE generates monodispersed droplets, diameter is measured in a subset of wells selected from a 384 well plate. All wells yield monodispersed emulsions, and minimal difference in droplet size between wells was observed (FIG. 2A). The overall droplet dispersity is 4±1%, which is superior to parallel droplet makers (5-10%) and slightly worse than single drop makers (1-3%). For PTE, sample encapsulation depends on aqueous shell volume. For plate PTE, the average diameter is 70±4 μm, higher than the polyacrylamide templating particles average of 67±2 μm; this corresponds to an aqueous shell of 3 μm (22 pL). The templating particles are generated with microfluidics, although commercial particles can also be used though generally have lower monodispersity.

The PTE process works over a range of sample volumes, from microliters to above milliliters (M. N. Hatori, Set al., Anal. Chem., 2018, 90, 9813-9820) If a library of smaller reagent diversity is desired, 96 well plates can be used, which can accommodate a larger working volume per well of 200 μL. The resulting droplets are monodispersed, with diameters of 53±2 μm with 49±2 μm particles (FIG. 2B). Alternatively, for applications in which higher diversity libraries are desired, 1536 well plates can be used; the smaller wells working volumes of 12 μL, but yield droplets of similar dispersity, 51±4 μm with 49±2 μm particles (FIG. 2B). These results demonstrate that PTE is applicable to common well plate formats and that the resultant droplet dimensions depend on particle size, not well size or number, illustrating the potential to produce high diversity droplet libraries.

### Removal of Satellites With Oil Washes

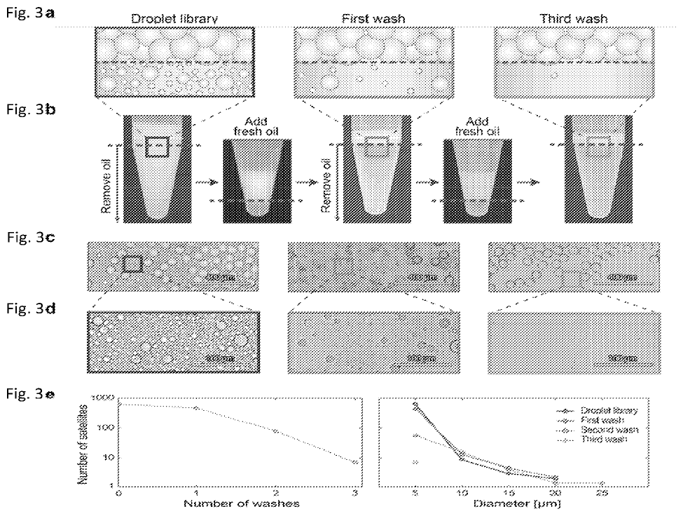

Satellites are sub-micron to micron droplets generated in PTE that can interfere with imaging and usage of the emulsions in microfluidic devices. Because they are much smaller than the templated droplets and do not contain particles, they can be removed by exploiting their lower buoyancy. After an emulsion is mixed, the droplets, which have a lower mass density than the carrier oil float (cream) to the surface with a rate that depends on buoyancy and the opposing viscous drag of the oil. Because buoyancy depends on the volume of the droplet and drag on its surface area (R. Pal, Fluids, 2019, 4, 186), big drops cream faster than small ones. Thus, the emulsion can be enriched for large drops by homogenizing, waiting for the large drops to cream, and removing the lower oil phase enriched in satellites (FIG. 3A). 30 seconds allows for droplets with diameters 65-75 μm to cream (FIG. 3B). The lower oil phase is laden with satellites and removed with a pipette (FIG. 3C). Additional oil can be added, and the process repeated, to successively enrich for the templated droplets (FIG. 3D). The resultant emulsions are nearly free of satellites and compatible with microfluidic processing.

### Generation of a Fluorescent Droplet Library

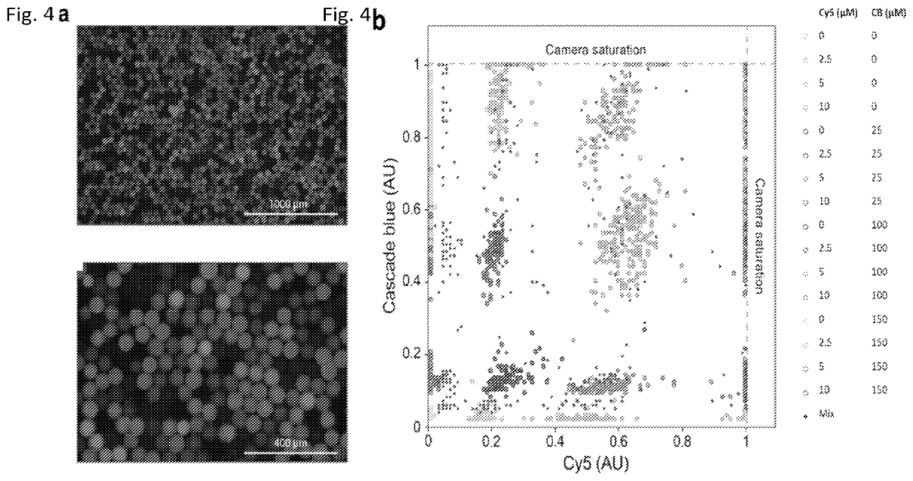

To demonstrate that plate PTE can produce droplet libraries containing distinct reagents, a library of 16 emulsions encoded with different fluorescent dyes was created. Two dyes at four different concentrations were mixed in separate wells, emulsify by PTE, and measure the resultant fluorescence of the emulsions separately, establishing the 16 baseline profiles. Next, the droplets were pooled into a single library and image them (FIG. 4A). The results show that the droplets have similar fluorescence before and after pooling, with the 16 dye combinations observable as distinct clusters when plotted (FIG. 4B). The scatter in the data is due to the challenge of obtaining accurate fluorescence profiles for packed emulsions imaged over a wide field of view, while the increased fluorescence of the 0 μM Cy5 and 0 μM Cascade Blue droplets is likely due to micelle-mediated transport (Y. Skhiri, et al., Soft Matter, 2012, 8, 10618).

### Sorting a Fluorescent Droplet Library

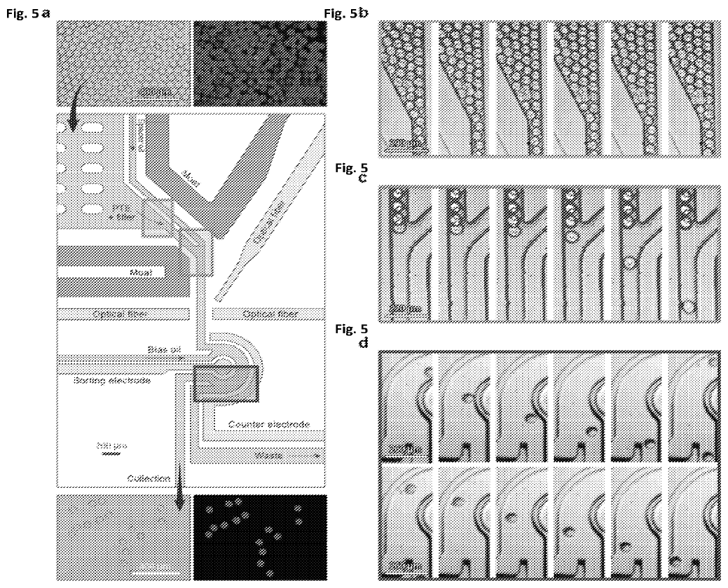

Fluorescence-activated droplet sorting is an important operation in droplet microfluidics that requires a narrow size distribution to be reliable (J. Fattaccioli, et al., Soft Matter, 2009, 5, 2232; and X. Wang, et al., Microfluid Nanofluid, 2018, 22). It is also one of the most difficult droplet microfluidic devices to design and operate due to the sensitivity of the sorting process on polydispersity. Thus, to demonstrate the utility of plate PTE libraries for microfluidic processing, we characterize the ability to sort them. A droplet library was prepared consisting of two fluorescent profiles generated across 16 different wells in a 384 well plate and pool the results into a library and run through a microfluidic sorter selecting only Cy5 positive droplets (FIG. 5A). Due to their uniformity, the droplets self-assemble into a regular lattice packing (FIG. 5B) which, subsequently, allows for reliable single droplet spacing (FIG. 5C). The individual droplets pass the excitation and emission fiber optics, which we use to select the Cy5 positives for sorting (FIG. 5D top), while Cascade Blue droplets are sent to waste (FIG. 5D bottom). This shows that plate PTE droplets are compatible with droplet sorting and illustrates the compatibility of these novel emulsions with microfluidic processing.

### Droplet Digital PCR in Well Plates

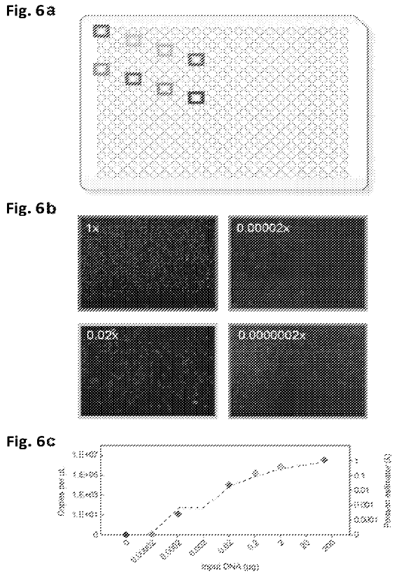

For plate PTE emulsions to be of value, they must be compatible with molecular biology reactions. To illustrate this, plate PTE was used to perform digital droplet polymerase chain reaction (ddPCR), a common application of droplet microfluidics. A dilution series of yeast DNA was prepared in a 384 well plate (FIG. 6A). The quantification of positive droplets provides a measure of the target DNA within that well (FIG. 6B). The analysis of the positive droplets and dilution provides an estimate of the DNA concentration in the sample (FIG. 6C). From this analysis we estimate a concentration of 30 million copies per μL for the undiluted sample. For a yeast genome, 30 million copies of the target correspond to 390 μg per mL, compared to the labelled concentration of 105 μg per mL. Successful execution of droplet digital PCR illustrates the compatibility of plate PTE with common molecular biology reactions.

As shown herein, particle-templated emulsification in well plates produces high-diversity droplet libraries compatible with microfluidics. This approach requires minimal equipment and expertise and is independent of the number and geometry of wells, producing monodisperse libraries with 30 seconds of plate vortexing. Because the approach uses standard well plates, it is compatible with automated pipetting and robotic liquid handling, streamlining production of droplet libraries. With plate PTE, applications requiring the combining of thousands of distinct chemical reagents, such as combinatorial drug screening and synthesis, are practical for the first time. These high value applications have been largely ignored due to the challenge of generating droplet libraries of sufficient diversity and quality. With plate PTE, these libraries can now be easily produced, expanding the application space available to this important field.

### Conversion of Single PTE Emulsions Into Double Emulsions

To demonstrate the ability to convert single PTE emulsions into double emulsions and manipulate double emulsions, ddPCR was performed on yeast genomic DNA and sorted via FACS. Preparation of the single emulsion for ddPCR was performed using particle templated emulsification. Briefly, 100 uL of a hard packed polyacrylamide pellet was mixed with 4 uL of 50 uM TaqMan probe, 4 uL of 50 uM TaqMan primer mix, 2.25 uL of 100 uM Cy5 background dye, 2.25 uL Triton X100, and 112.5 uL of 2×Platinum Master Mix. The mixture was then centrifuged at 6000 rcf for 1 min and the supernatant removed. 1 uL of yeast genomic DNA was added to the polyacrylamide pellet and reagents. Particle templated emulsification was then performed after the addition of 200 uL of 2% 008-fluoro-surfacant in Novec-7500 and thermocycled. Following PTE, the oil phase was replaced with 50 uL of 5% 008-fluoro-surfactant in FC40 oil.

Conversion from a single to double emulsion was achieved by the addition of a carrier solution and agitation. Briefly, a carrier phase consisting of 40 mM KCl, 12.5 mM Tris-HCl, 1.5 mM MgCl2, 10% PEG35k, 4% Tween20, 1% Pluronic F68 was prepared. 300 uL of carrier phase was then added to the single emulsion generated with PTE. Agitation via tapping and vortexing with the carrier phase results in a second encapsulation forming water-in-oil-in-water emulsions. The addition of 100 uL 5M NaCl results in osmotic shrinking of the inner aqueous phase of the double emulsion. The resulting double emulsions were then loaded into a BD FACSAria sorter with the resulting readout used to detect positive double emulsions and sort them.

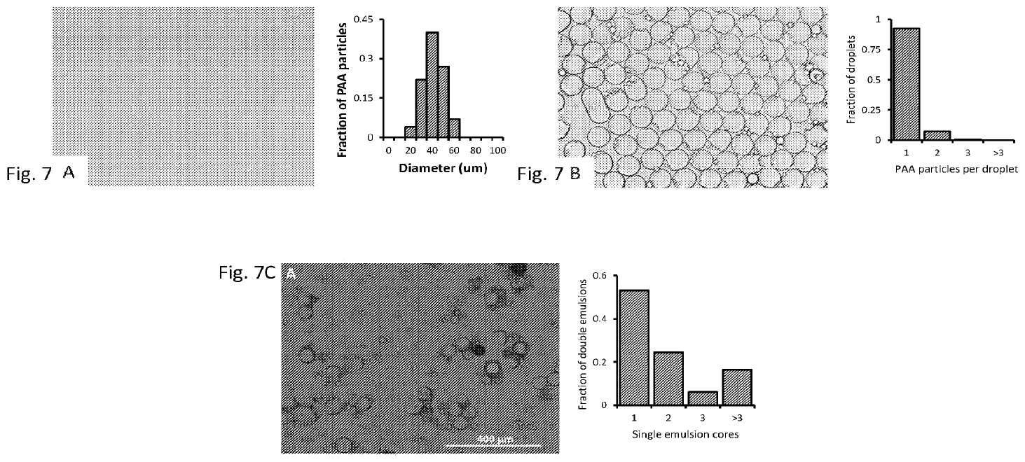

FIG. 7 shows the generation of double emulsions using particle templated emulsification: (A) Particles used in PTE to generate emulsions; (B) Single emulsions generated using PTE; and (C) Conversion to double emulsions using single PTE emulsions.

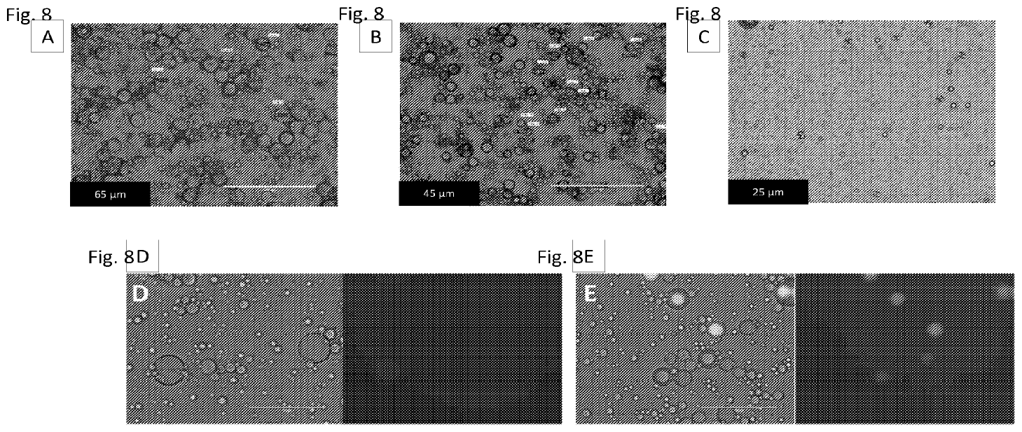

As shown in FIG. 8, PTE double emulsions size and stability is affected by droplet size. (A) Initial generation of PTE double emulsions results in a relatively thin oil layer separating the inner aqueous droplet contents from the outer aqueous phase. (B) Addition of salt to the outer buffer results in a hypertonic solution causing the double emulsion to shrink. (C) Further addition of salt to the outer solution causes the droplet to continue to shrink. The smaller double emulsions are significantly more stable than the original emulsions. (D) and (E) For PTE double emulsions, shrinking the double emulsion results in a significantly brighter signal.

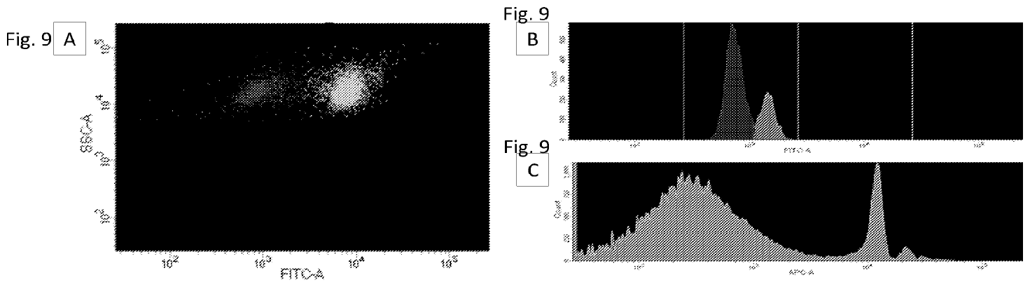

As shown in FIG. 9, PTE double emulsions can be sorted via FACS and avoids the use of custom made microfluidic sorters. (A) FACS side scatter vs FITC identifies two unique populations of droplets. (B) distinct populations of FITC positive (ddPCR positive) and FITC negative double emulsions can be identified via FACS. (C) Additionally, fractions of double emulsions containing single, double, and triple cores can be identified using FACS. Gating and sorting based on these populations is possible using the FACS.

### Methods

### Templating Particle Fabrication

Polyacrylamide templating particles were prepared using microfluidic droplet makers. The acrylamide solution consists of 6.2% acrylamide, 0.32% N,N′-mthylenebis (acrylamide), and 0.3% ammonium persulfate. The oil consists of a 2% (w/w) fluorosurfactant and 1% (w/v) N,N,N,N-tetramethylethylenediame (TEMED) in hydrofluoroether (HFE). The microfluidic drop maker is run at 250 μL/h and 500 μL/h for acrylamide and oil inputs respectively. The resulting droplets are incubated at room temperature overnight. An equal volume of 20% (v/v) perfluoro-1-octanol (PFO) in HFE is added to the droplets to de-emulsify. Centrifuging the solution at 2000×g for 2 min separates the oil and hydrogel particles. After removal of the oil, the particles are washed twice using 3 volumes of 2% (v/v) sorbitan monooleate in hexane. The supernatant is removed after centrifugation at 3000×g for 3 min. The particles were then washed twice with TEBST (20 mM Tris-HCl pH 8.0, 274 mM NaCl, 5.4 mM KCl, 20 mM EDTA, 0.2% Triton X-100), removing the supernatant after centrifugation at 3000×g for 3 min after each wash. Particles were resuspend with 2% Triton X-100 in phosphate buffered saline and store at 4° C. until use.

### Well Plate Particle-Templated Emulsification

Well plate assembly prior to emulsification consists of adding particles, sample, then oil. Depending on well volume 38 μL, 8 μL, and 3 μL of hard packed particles is added to each well for 96, 384, and 1532 well plates respectively. Accordingly, to each well 2 μL of phosphate buffered saline were added for 96 and 384 well plates and 1 μL of phosphate buffered saline for 1532 well plates. The well plate was sealed and shaken for 5 min to mix. After centrifuging at 200×g for 1 min and removing the seal, we add 80 μL, 20 μL, and 8 μL 2% (w/w) fluorosurfactant in HFE to each well. After resealing the plate, the plate is vortexed for 30 seconds using a vortexer with a flat head. The plate was centrifuged at 200×g for 1 min then remove the seal to prevent any cross contamination. Using a wide bore pipette tip, emulsions were collected from the wells to individually examine or pool into a droplet library. Resulting measurements are performed using an EVOS FL Auto and ImageJ software.

### Oil Washes

For oil washes, emulsions were collected into a 1.5 mL micro centrifuge tube. After 1 minute, a strong backlight at an oblique angle is used to identify the interface between oil and emulsion. Using a gel loading pipette tip, the bottom oil phase is removed. Fresh 2% (w/v) fluorosurfactant in HFE is then added. After the addition of fresh oil, an aliquot is imaged using an EVOS FL Auto and ImageJ software to determine the size and number of satellites. This wash is repeated two additional times.

### Fluorescent Droplet Library

Preparation of a fluorescent droplet library is performed in a 384 well plate using 16 different combinations of Cy5 and Cascade Blue. As before, 8 μL of hard packed particles is added to each well in the 384 well plate. 0 μM, 2.5 μM, 5 μM and 10 μM Cy5 with 0 μM, 25 μM, 100 μM, and 150 μM Cascade Blue was the mixed to create 16 different solutions of Cy5 and Cascade Blue. 2 μL of each solution was added to a well. The plate is sealed and placed on a shaker for 5 min to mix. After centrifuging at 200×g for 2 min the seal was removed and 20 μL 2% (w/v) fluorosurfactant in HFE was added to each well. The plate is resealed and vortexed for 30 seconds. The emulsified plate is then centrifuged, and the seal removed. Using a wide bore pipette tip, 2 μL of emulsions is removed and imaged using an EVOS FL Auto to establish a fluorescent baseline for each well. Then all wells are collected into a droplet library and imaged. The resulting fluorescent profile of the baselines and droplet library is evaluated using ImageJ.

### Microfluidic Sorting

To determine the compatibility of well plate PTE with microfluidic sorting, a fluorescent droplet library was prepared and sorted. Here the droplet library was prepared by pooling 8 wells containing either 2.5 μM Cy5 with 150 μM Cascade Blue or 10 μM Cy5 with 25 μM Cascade Blue. The sorter device is prepared by molding poly (dimethylsiloxane) (PDMS) over a SU-8 master. The SU-8 master is prepared using 20, 80, and 150 μm photolithographic layers. The PDMS is cured at 65° C. for 2 h. Inlets are punched using a 0.75 mm biopsy punch. Devices are then plasma bonded to glass slides and treated using AquaPel. A 105 um diameter multimode excitation fiber with an NA of 0.22 and a 200 μm diameter multimode detection fiber with an NA of 0.39 are inserted into the corresponding guide channel. Lasers with 405 and 640 nm are coupled to the excitation fiber, while the emission fiber is columnated, passes through bandpass filters, appropriate dichroic mirror to PMTs. The sorting electrode is filled with 5 M NaCl and connected to a voltage amplifier and a function generator. The counter electrode is filled with 5 M NaCl and grounded. 0.2% w/w ionic krytox surfactant in HFE is used for input to both spacer and bias channels. Syringe pumps (New Era) provide constant flow rates of 1000 μL/h for both spacer and bias input. The waste outlet in the microfluidic device was run at −3000 μL/h. The droplet library is then reinjected with a flow rate of 50 μL/h. Positive droplets are selected using the Lab View software analysis of droplet fluorescence. Sorting occurs by applying a high frequency pulse applied to the sorting electrode, with parameters 10 to 20 kHz, 50 to 100 cycles, and 0.5 to 1.0 kV. Both collected droplets and the droplet library are imaged using an EVOS FL Auto.

### Digital Droplet PCR

To perform droplet digital PCR, a mixture was prepared containing hard packed particles with master mix and probes. Specifically, the mixture contains 32% v/v hard packed particles, 0.8 μM forward primer, 0.8 μM reverse primer, 0.8 μM TaqMan probe, 1×Platinum Taq Master Mix, and 2% Triton X100. The mixture was incubated with gentle agitation at room temperature for 5 minutes to allow for diffusion into the particles. The mixture was then centrifuged at 6000×g for 2 minutes and the supernatant removed. A wide bore pipette tip was used to transfer 10 μL of the hard packed particles to each well in a 384 well plate. 1 μL of a yeast genomic DNA dilution series (1×, 0.02×, 0.0002×, 0.00002×, 0.000002×, 0.0000002×, 0.00000002×, and 0×) was added to each well. The plate was then sealed, briefly vortexed, then 20 μL 2% (w/v) fluorosurfactant in HFE was added to each well. The plate was then sealed a second time and vortexed for 30 seconds for particle-templated emulsification. The plate was then loaded into a BioRad CFX384 and thermocycled 2 minutes at 95° C. followed by 34 cycles of 95° C. for 10 seconds, 60° C. for 30 seconds, and 72° C. for 2 minutes before a final extension at 72° C. for 2 minutes. Using a wide bore pipette tip 10 μL from each well was collected and imaged using a EVOS FL Auto.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of generating a droplet library, comprising:
(a) preparing a reagent library comprising combining in single wells of a multi-well plate (i) a collection of particles; (ii) an immiscible carrier; and (iii) a solution comprising a plurality of reagents;
(b) agitating the combined solution of (a) under conditions that allow the encapsulation of the solution comprising a plurality of reagents into droplets, thereby forming a monodisperse emulsion; and
(c) optionally pooling the droplets formed in the individual wells of (b) into a vessel;
thereby generating a droplet library.

2. The method of claim 1, wherein the multi-well plate comprises 4, 6, 8, 12, 24, 48, 96, 384, or 1536 wells.

3. The method of any one of claims 1-2, wherein the total aqueous volume in an individual well is between approximately 3-100 μl.

4. The method of any one of claims 1-2, wherein the droplet diameter is approximately 20-200 um.

5. The method of claim 2, wherein the multi-well plate comprises 96 wells, wherein the total aqueous volume is approximately 200 μl, and wherein the droplet diameter is approximately 60-70 um.

6. The method of claim 2, wherein the multi-well plate comprises 384 wells, wherein the total aqueous volume is approximately 10 μl, and wherein the droplet diameter is approximately 60-70 um.

7. The method of claim 2, wherein the multi-well plate comprises 1536 wells, wherein the total aqueous volume is approximately 3 μl, and wherein the droplet diameter is approximately 60-70 um.

8. The method of any of the preceding claims, wherein the particles are selected from the group consisting of hydrogel beads, plastic beads, glass beads, ceramic beads, and magnetic beads.

9. The method of claim 8, wherein the particles are hydrogel beads comprising acrylamide.

10. The method of any of the preceding claims, wherein the immiscible carrier is an oil.

11. The method of claim 10, wherein the oil comprises a fluorosurfactant and N,N,N,N-tetramethylethylenediame in hydrofluoroether.

12. The method of any of the preceding claims, wherein prior to agitating, the multi-well plate is sealed.

13. The method of claim 12, wherein the agitating comprises mixing the reagents by pipetting, shaking by hand, stirring, beating, bubbling, vortexing and sonicating.

14. The method of any of the preceding claims, wherein each well of the multi-well plate comprises a solution comprising a plurality of reagents and wherein at least one reagent is unique to each individual well.

15. The method of any of the preceding claims, wherein the plurality of reagents comprises a sample comprising biomolecules.

16. The method of claim 15, wherein the sample is obtained from a human subject.

17. The method of claim 15, wherein the sample is a saliva, blood, urine, or tissue sample.

18. The method of claim 15, wherein the sample comprises a plurality of cells selected from the group consisting of a virus or virus particle, a bacterial cell, a yeast cell, a parasitic cell, or a human cell.

19. The method of claim 18, wherein the sample has not undergone purification steps prior to combining in a well of step (a).

20. The method of any of the preceding claims, wherein the plurality of reagents comprises at least one nucleic acid.

21. The method of claim 20, wherein the plurality of reagents comprises reagents suitable for amplifying the nucleic acid.

22. The method of claims 21, wherein the plurality of reagents comprises reagents suitable for a polymerase chain reaction (PCR) or reagents suitable for a loop-mediated isothermal amplification (LAMP) reaction or reagents for a nucleic acid sequence-based amplification (NASBA) reaction, and optionally comprising a lysing reagent.

23. The method of claim 23, wherein the lysing reagent, when present, is SDS.

24. The method of any one of claims 21-23, wherein the droplet is incubated under conditions that allow amplification, and the nucleic acid is amplified by a method selected from the group consisting of PCR, RT-PCR, qPCR, digital droplet PCR (ddPCR), LAMP and NASBA.

25. The method of claim 18, wherein each droplet formed in step (b) comprises a single cell.

26. The method of any of the preceding claims, wherein each droplet comprises a barcode.

27. The method of any of the preceding claims, further comprising the step of removing satellite droplets.

28. The method of any of the preceding claims, further comprising the step of sorting the droplets.

29. The method of any of the preceding claims, further comprising the step of converting single water-in-oil droplets that are generated in the agitating step (b) into double water-in-oil-in-water droplets, wherein said double water-in-oil-in-water droplets are compatible with aqueous based manipulation.
